(19) United States
(12) Patent Application Publication
(10) Pub. No.: US 2018/0280562 A1
(43) Pub. Date: Oct. 4, 2018

(54) Fibrous Structures Comprising Sensates

(71) Applicant: The Procter & Gamble Company, Cincinnati, OH (US)

(72) Inventors: LaTisha Evette Salaam-Zayid, Cincinnati, OH (US); Koti Tatachar Sreekrishna, Mason, OH (US)

(73) Assignee: The Procter & Gamble Company

(21) Appl. No.: 15/924,441

(22) Filed: Mar. 19, 2018

Related U.S. Application Data
(60) Provisional application No. 62/478,110, filed on Mar. 29, 2017.

(51) Int. Cl. A61L 15/44; A61L 15/42; A61L 15/28

(52) U.S. Cl. CPC A61L 15/44; A61L 2300/40; A61L 15/28; A61L 15/42

(57) ABSTRACT

Fibrous structures, for example sanitary tissue products, such as toilet tissue, containing one or more sensates, more particularly one or more TRPM8 receptor triggering agents, a sensate composition, for example surface softening composition, comprising one or more TRPM8 receptor triggering agents, and methods for making same are provided.

### FIELD OF THE INVENTION

The present invention relates to fibrous structures, for example sanitary tissue products, such as toilet tissue, comprising one or more TRP receptor agents, more particularly one or more TRP receptor triggering agents (also referred to as “agonists”) (“sensates” as used herein) and optionally, one or more TRP receptor blocking agents (also referred to as “antagonists”), a sensate composition, for example a surface softening composition, comprising one or more TRP receptor agents, such as one or more TRP receptor triggering agents (“sensates”), for example one or more TRPM8 receptor triggering agents, and methods for making same.

### BACKGROUND OF THE INVENTION

Fecal residue left behind during a bowel movement cleaning event leads to consumer discomfort around feeling clean and fresh. This discomfort can escalate to feelings of fear, shame and incompetence, due to the presence of residual soil (fecal matter) and the sensorial impacts that result from the residual fecal matter; namely, lack of a fresh feel on a user's skin during and/or after a bowel movement.

The physiological tactile sensation and sensory perception of what is clean and fresh continues to be an untapped, undefined, and unmet need for consumers of sanitary tissue products, such as toilet tissue. There are several problems that exist when formulators attempt to provide a sensation of freshness and cleaning to a user of toilet tissue by triggering TRP receptors, for example by triggering TRPM8 receptors that provide a cooling sensation, and optionally blocking other TRP receptors, such as TRPA1, TRPV, TRPV1, TRPA1V1, TRPV2, TRPV3, TRPV4 receptors that provide sensations such as warming, burning, and tingling when triggered.

Due to the relative sensitive nature of anal skin and the skin surrounding the anal region both of which contain TRP receptors, formulators need to identify suitable TRP receptor agents, including combinations thereof, and levels of such TRP receptor agents that deliver an overall pleasant experience to a consumer of such toilet tissue comprising one or more TRP receptor agents. Non-limiting examples of TRP receptor agents include TRP receptor triggering agents (“sensates”), for example TRPM8 receptor triggering agents. For purposes of the present invention, sensates are materials which their predominant effect on skin tissue is to elicit a sensation, for example a warming, cooling, burning, and/or tingling sensation, by triggering one or more TRP receptors, for example eliciting a cooling sensation by triggering the TRPM8 receptor. The TRP receptor triggering agents (“sensates”) trigger (“activate”) a TRP receptor, for example by the TRP receptor triggering agent (“sensate”) binding to the TRP receptor to produce a tactile, physiological response, in other words to elicit a sensation that the consumer experiences, pleasant or unpleasant.

Examples of negatives encountered by formulators attempting to include one or more TRP receptor triggering agents (“sensates”) on fibrous structures, especially on user contacting surfaces of fibrous structures, for example sanitary tissue products, such as toilet tissue are described below. For example, upon contacting anal skin and/or skin surrounding the anal region with toilet tissue comprising one or more TRP receptor triggering agents (“sensates”) during use by a consumer an unpleasant and/or painful cooling and/or warming and/or burning and/or extremely cold sensations may be experienced by consumers of the toilet tissue due to types and/or levels of TRP receptor triggering agents (“sensates”) present on the toilet tissue; unpleasant smell, such as a chemical smell exhibited by the TRP receptor triggering agents (“sensates”) present on the toilet tissue; and/or unacceptable longevity of sensation elicited by the TRP receptor triggering agents (“sensates”) on a consumer's skin, for example due to undesirable TRP receptors, such as TRPA1, TRPV, TRPV1, TRPA1V1, TRPV2, TRPV3, TRPV4 receptors being triggered by the TRP receptor triggering agents (“sensates”).

One problem faced by formulators of fibrous structures, for example sanitary tissue products, such as toilet tissue, is providing fibrous structures that provide a sensation to a consumer, for example after wiping one's skin, for example anal skin, with the fibrous structures such that the consumer experiences an acceptable and/or pleasant physiological tactile sensation and/or sensory perception of being clean and/or fresh, not an unacceptable and/or unpleasant experience as described above.

There is a need for a fibrous structure, for example sanitary tissue product, such as a toilet tissue, comprising one or more TRP receptor triggering agents (“sensates”) that delivers a consumer acceptable and/or consumer desirable sensation, for example a pleasant cooling sensation, and methods for making such fibrous structure.

### SUMMARY OF THE INVENTION

The present invention fulfills the needs described above by providing a fibrous structure, for example a sanitary tissue product, such as a toilet tissue, comprising one or more TRP receptor triggering agents (“sensates”) that deliver a consumer acceptable and/or consumer desirable sensation.

One solution to the problem described above is to provide a fibrous structure, for example a sanitary tissue product, such as a toilet tissue, comprising one or more TRP receptor triggering agents (“sensates”), such as one or more TRPM8 receptor triggering agents, and optionally, one or more TRP receptor blocking agents that result in a user of such a fibrous structure experiencing a pleasant and/or desirable physiological tactile sensation and/or sensory perception after contacting the user's skin with such a fibrous structure.

In one example of the present invention, a fibrous structure comprising a sensate composition comprising one or more TRP receptor triggering agents (“sensates”), such as one or more TRPM8 receptor triggering agents, for example a non-sublimable TRPM8 receptor triggering agent and/or a TRPM8 receptor triggering agent that does not create an olfactory response, and optionally, one or more TRP receptor blocking agents, is provided. Non-limiting examples of TRPM8 receptor triggering agents that create an olfactory response include menthol and camphor.

In another example of the present invention, a sensation composition comprising a surface softening composition comprising one or more TRP receptor triggering agents (“sensates”), such as one or more TRPM8 receptor triggering agents, for example a non-sublimable TRPM8 receptor triggering agent and/or a TRPM8 receptor triggering agent that does not create an olfactory response, and one or more surface softening agents, and optionally, one or more TRP receptor blocking agents, is provided.

In yet another example of the present invention, a method for making a fibrous structure, the method comprising the step of applying a sensate composition comprising one or more TRP receptor triggering agents (“sensates”), such as one or more TRPM8 receptor triggering agents, for example a non-sublimable TRPM8 receptor triggering agent and/or a TRPM8 receptor triggering agent that does not create an olfactory response, for example WS23, and optionally, one or more TRP receptor blocking agents, to a fibrous structure, is provided.

In even another example of the present invention, a method for making a fibrous structure, the method comprising the step of applying a sensate composition comprising a surface softening composition comprising one or more TRP receptor triggering agents (“sensates”), such as one or more TRPM8 receptor triggering agents, for example a non-sublimable TRPM8 receptor triggering agent and/or a TRPM8 receptor triggering agent that does not create an olfactory response, and one or more surface softening agents, and optionally, one or more TRP receptor blocking agents to a fibrous structure, is provided.

In even yet another example of the present invention, a single- or multi-ply sanitary tissue product, for example toilet tissue and/or facial tissue, comprising one or more fibrous structures of the present invention, is provided.

In still another example of the present invention, a method for eliciting a sensation on a user's skin, the method comprising the step of contacting a user's skin, for example anal skin, which contains one or more TRP receptors, for example a TRPM8 receptor, with a fibrous structure comprising one or more TRP receptor triggering agents (“sensates”), such as one or more TRPM8 receptor triggering agents, for example a non-sublimable TRPM8 receptor triggering agent and/or a TRPM8 receptor triggering agent that does not create an olfactory response, and optionally one or more TRP receptor blocking agents such that a sensation is elicited, for example from the TRPM8 receptor, and optionally, one or more TRP receptors, for example one or more of TRPA1, TRPV, TRPV1, TRPA1V1, TRP2, TRP3, and TRPV4 receptors are blocked (or dampened), is provided.

The present invention provides a fibrous structure, for example a sanitary tissue product, such as toilet tissue, comprising one or more TRP receptor triggering agents (“sensates”), for example one or more TRPM8 receptor triggering agents, a sensate composition, for example a surface softening composition, comprising one or more TRP receptor triggering agents (“sensates”), for example one or more TRPM8 receptor triggering agents, and optionally, one or more TRP receptor blocking agents, methods for making same, and method for eliciting a sensation on a user of the fibrous structure.

### DETAILED DESCRIPTION OF THE INVENTION

“Sanitary tissue product” as used herein means a soft, low density (i.e. <about 0.15 g/cm³) article comprising one or more fibrous structure plies according to the present invention, wherein the sanitary tissue product is useful as a wiping implement for post-urinary and post-bowel movement cleaning (toilet tissue), for otorhinolaryngological discharges (facial tissue), and multi-functional absorbent and cleaning uses (absorbent towels). The sanitary tissue product may be convolutedly wound upon itself about a core or without a core to form a sanitary tissue product roll, which may be packaged alone or together with one or more other rolls in known packaging materials, for example polywrap and/or film for sale, such as retail sales.

The sanitary tissue products and/or fibrous structures of the present invention may exhibit a basis weight of greater than 15 g/m² to about 120 g/m² and/or from about 15 g/m² to about 110 g/m² and/or from about 20 g/m² to about 100 g/m² and/or from about 30 to 90 g/m². In addition, the sanitary tissue products and/or fibrous structures of the present invention may exhibit a basis weight between about 40 g/m² to about 120 g/m² and/or from about 50 g/m² to about 110 g/m² and/or from about 55 g/m² to about 105 g/m² and/or from about 60 to 100 g/m².

The sanitary tissue products of the present invention may exhibit a sum of MD and CD dry tensile strength of greater than about 59 g/cm (150 g/in) and/or from about 78 g/cm to about 394 g/cm and/or from about 98 g/cm to about 335 g/cm. In addition, the sanitary tissue product of the present invention may exhibit a sum of MD and CD dry tensile strength of greater than about 196 g/cm and/or from about 196 g/cm to about 394 g/cm and/or from about 216 g/cm to about 335 g/cm and/or from about 236 g/cm to about 315 g/cm. In one example, the sanitary tissue product exhibits a sum of MD and CD dry tensile strength of less than about 394 g/cm and/or less than about 335 g/cm.

In another example, the sanitary tissue products of the present invention may exhibit a sum of MD and CD dry tensile strength of greater than about 196 g/cm and/or greater than about 236 g/cm and/or greater than about 276 g/cm and/or greater than about 315 g/cm and/or greater than about 354 g/cm and/or greater than about 394 g/cm and/or from about 315 g/cm to about 1968 g/cm and/or from about 354 g/cm to about 1181 g/cm and/or from about 354 g/cm to about 984 g/cm and/or from about 394 g/cm to about 787 g/cm.

The sanitary tissue products of the present invention may exhibit an initial sum of MD and CD wet tensile strength of less than about 78 g/cm and/or less than about 59 g/cm and/or less than about 39 g/cm and/or less than about 29 g/cm.

The sanitary tissue products of the present invention may exhibit an initial sum of MD and CD wet tensile strength of greater than about 118 g/cm and/or greater than about 157 g/cm and/or greater than about 196 g/cm and/or greater than about 236 g/cm and/or greater than about 276 g/cm and/or greater than about 315 g/cm and/or greater than about 354 g/cm and/or greater than about 394 g/cm and/or from about 118 g/cm to about 1968 g/cm and/or from about 157 g/cm to about 1181 g/cm and/or from about 196 g/cm to about 984 g/cm and/or from about 196 g/cm to about 787 g/cm and/or from about 196 g/cm to about 591 g/cm.

The sanitary tissue products of the present invention may exhibit a density (based on measuring caliper at 95 g/in²) of less than about 0.60 g/cm³ and/or less than about 0.30 g/cm³ and/or less than about 0.20 g/cm³ and/or less than about 0.10 g/cm³ and/or less than about 0.07 g/cm³ and/or less than about 0.05 g/cm³ and/or from about 0.01 g/cm³ to about 0.20 g/cm³ and/or from about 0.02 g/cm³ to about 0.10 g/cm³.

The sanitary tissue products of the present invention may be in the form of sanitary tissue product rolls. Such sanitary tissue product rolls may comprise a plurality of connected, but perforated sheets of fibrous structure, that are separably dispensable from adjacent sheets.

In another example, the sanitary tissue products may be in the form of discrete sheets that are stacked within and dispensed from a container, such as a box.

The fibrous structures and/or sanitary tissue products of the present invention may comprise additives such as surface softening agents, for example silicones, quaternary ammonium compounds, aminosilicones, lotions, and mixtures thereof, temporary wet strength agents, permanent wet strength agents, bulk softening agents, wetting agents, latexes, especially surface-pattern-applied latexes, dry strength agents such as carboxymethylcellulose and starch, and other types of additives suitable for inclusion in and/or on sanitary tissue products.

“Fibrous structure” as used herein means a structure that comprises one or more fibers and/or filaments. In one example, a fibrous structure at least comprises a plurality of fibers. In one example, the fibrous structure may comprise a plurality of wood pulp fibers. In another example, the fibrous structure may comprise a plurality of non-wood pulp fibers, for example plant fibers, synthetic staple fibers, and mixtures thereof. In still another example, in addition to pulp fibers, the fibrous structure may comprise a plurality of filaments, such as polymeric filaments, for example thermoplastic filaments such as polyolefin filaments (i.e., polypropylene filaments) and/or hydroxyl polymer filaments, for example polyvinyl alcohol filaments and/or polysaccharide filaments such as starch filaments. In one example, a fibrous structure according to the present invention means an orderly arrangement of fibers alone and with filaments within a structure in order to perform a function. Non-limiting examples of fibrous structures of the present invention include paper.

Non-limiting examples of processes for making fibrous structures include known wet-laid papermaking processes and air-laid papermaking processes. Such processes typically include steps of preparing a fiber composition, oftentimes referred to as a fiber slurry in wet-laid processes, either wet or dry, and then depositing a plurality of fibers onto a forming wire or belt such that an embryonic fibrous structure is formed, drying and/or bonding the fibers together such that a fibrous structure is formed, and/or further processing the fibrous structure such that a finished fibrous structure is formed. For example, in typical papermaking processes, the finished fibrous structure is the fibrous structure that is wound on the reel at the end of papermaking, but before converting thereof into a sanitary tissue product.

Non-limiting types of fibrous structures according to the present invention include conventionally felt-pressed fibrous structures; pattern densified fibrous structures; and high-bulk, uncompacted fibrous structures. The fibrous structures may be of a homogeneous or multilayered (“layered” meaning two or three or more layers) construction; and the sanitary tissue products made therefrom may be of a single-ply or multi-ply construction.

The fibrous structures may be post-processed, such as by embossing and/or calendaring and/or folding and/or printing images thereon.

The fibrous structures may be through-air-dried fibrous structures or conventionally dried fibrous structures.

The fibrous structures may be creped or uncreped. In one example, the fibrous structures may be belt-creped and/or fabric creped.

Non-limiting examples of processes for making fibrous structures include known wet-laid papermaking processes, for example conventional wet-pressed papermaking processes and through-air-dried papermaking processes, and air-laid papermaking processes. Such processes typically include steps of preparing a fiber composition in the form of a suspension in a medium, either wet, more specifically aqueous medium, or dry, more specifically gaseous, i.e. with air as medium. The aqueous medium used for wet-laid processes is oftentimes referred to as a fiber slurry. The fibrous slurry is then used to deposit a plurality of fibers onto a forming wire, fabric, or belt such that an embryonic fibrous structure is formed, after which drying and/or bonding the fibers together results in a fibrous structure. Further processing the fibrous structure may be carried out such that a finished fibrous structure is formed. For example, in typical papermaking processes, the finished fibrous structure is the fibrous structure that is wound on the reel at the end of papermaking, often referred to as a parent roll, and may subsequently be converted into a finished product, e.g. a single- or multi-ply sanitary tissue product.

The fibrous structures of the present invention may be homogeneous or may be layered. If layered, the fibrous structures may comprise at least two and/or at least three and/or at least four and/or at least five layers of fiber and/or filament compositions.

In one example, the fibrous structure of the present invention consists essentially of fibers, for example pulp fibers, such as cellulosic pulp fibers and more particularly wood pulp fibers.

In another example, the fibrous structure of the present invention comprises fibers and is void of filaments.

In still another example, the fibrous structures of the present invention comprises filaments and fibers, such as a co-formed fibrous structure.

“Co-formed fibrous structure” as used herein means that the fibrous structure comprises a mixture of at least two different materials wherein at least one of the materials comprises a filament, such as a polypropylene filament, and at least one other material, different from the first material, comprises a solid additive, such as a fiber and/or a particulate. In one example, a co-formed fibrous structure comprises solid additives, such as fibers, such as wood pulp fibers, and filaments, such as polypropylene filaments.

“Fiber” and/or “Filament” as used herein means an elongate particulate having an apparent length greatly exceeding its apparent width, i.e. a length to diameter ratio of at least about 10. In one example, a “fiber” is an elongate particulate as described above that exhibits a length of less than 5.08 cm (2 in.) and a “filament” is an elongate particulate as described above that exhibits a length of greater than or equal to 5.08 cm (2 in.).

Fibers are typically considered discontinuous in nature. Non-limiting examples of fibers include pulp fibers, such as wood pulp fibers, and synthetic staple fibers such as polyester fibers.

Filaments are typically considered continuous or substantially continuous in nature. Filaments are relatively longer than fibers. Non-limiting examples of filaments include meltblown and/or spunbond filaments. Non-limiting examples of materials that can be spun into filaments include natural polymers, such as starch, starch derivatives, cellulose and cellulose derivatives, hemicellulose, hemicellulose derivatives, and synthetic polymers including, but not limited to polyvinyl alcohol filaments and/or polyvinyl alcohol derivative filaments, and thermoplastic polymer filaments, such as polyesters, nylons, polyolefins such as polypropylene filaments, polyethylene filaments, and biodegradable or compostable thermoplastic fibers such as polylactic acid filaments, polyhydroxyalkanoate filaments and polycaprolactone filaments. The filaments may be monocomponent or multicomponent, such as bicomponent filaments.

In one example of the present invention, “fiber” refers to papermaking fibers. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified fibrous structure. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

Natural papermaking fibers useful in the present invention include animal fibers, mineral fibers, plant fibers, and mixtures thereof. Animal fibers may, for example, be selected from the group consisting of: wool, silk, and mixtures thereof. Plant fibers may, for example, be derived from a plant selected from the group consisting of: wood, cotton, cotton linters, flax, sisal, abaca, hemp, hesperaloe, jute, bamboo, bagasse, kudzu, corn, sorghum, gourd, agave, loofah, and mixtures thereof. In one example the fibers comprise trichomes, such as trichomes obtained from Stachys bzyantina, for example trichomes from a Lamb's Ear plant.

Wood fibers; often referred to as wood pulps include chemical pulps, such as kraft (sulfate) and sulfite pulps, as well as mechanical and semi-chemical pulps including, for example, groundwood, thermomechanical pulp, chemi-mechanical pulp (CMP), chemi-thermomechanical pulp (CTMP), neutral semi-chemical sulfite pulp (NSCS). Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified and/or layered fibrous structure. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

The wood pulp fibers may be short (typical of hardwood fibers) or long (typical of softwood fibers). Non-limiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Non-limiting examples of long fibers include fibers derived from Pine, Spruce, Fir, Tamarack, Hemlock, Cypress, and Cedar. Softwood fibers derived from the kraft process and originating from more-northern climates may be preferred. These are often referred to as northern softwood kraft (NSK) pulps.

Synthetic fibers may be selected from the group consisting of: wet spun fibers, dry spun fibers, melt spun (including melt blown) fibers, synthetic pulp fibers, and mixtures thereof. Synthetic fibers may, for example, be comprised of cellulose (often referred to as “rayon”); cellulose derivatives such as esters, ether, or nitrous derivatives; polyolefins (including polyethylene and polypropylene); polyesters (including polyethylene terephthalate); polyamides (often referred to as “nylon”); acrylics; non-cellulosic polymeric carbohydrates (such as starch, chitin and chitin derivatives such as chitosan); and mixtures thereof.

“Ply” or “Plies” as used herein means an individual finished fibrous structure optionally to be disposed in a substantially contiguous, face-to-face relationship with other plies, forming a multiple ply finished fibrous structure product and/or sanitary tissue product. It is also contemplated that a single fibrous structure can effectively form two “plies” or multiple “plies”, for example, by being folded on itself.

“Surface of a fibrous structure” and/or “surface of sanitary tissue product” as used herein means that portion of the fibrous structure and/or sanitary tissue product that is exposed to the external environment. In other words, the surface of a fibrous structure and/or surface of a sanitary tissue product is that portion of the fibrous structure and/or sanitary tissue product that is not completely surrounded by other portions of the fibrous structure and/or sanitary tissue product.

“User Contacting Surface” as used herein means that portion of the fibrous structure and/or surface softening composition and/or lotion composition present directly and/or indirectly on the surface of the fibrous structure that is exposed to the external environment. In other words, it is that surface formed by the fibrous structure including any sensate composition and/or surface softening composition and/or lotion composition present directly and/or indirectly on the surface of the fibrous structure that contacts an opposing surface when used by a user. For example, it is that surface formed by the fibrous structure including any sensate composition and/or surface softening composition and/or lotion composition present directly and/or indirectly on the surface of the fibrous structure that contacts a user's skin, for example a user's anal skin or skin surrounding a user's anal region, when a user wipes his/her skin, for example after a bowel movement, with the fibrous structure of the present invention.

In one example, the user contacting surface, especially for a textured and/or structured fibrous structure, such as a through-air-dried fibrous structure and/or an embossed fibrous structure, may comprise raised areas and recessed areas of the fibrous structure. In the case of a through-air-dried, pattern densified fibrous structure the raised areas may be knuckles and the recessed areas may be pillows and vice versa. Accordingly, the knuckles may, directly and/or indirectly, comprise the sensate composition and/or the surface softening composition and/or lotion composition and the pillows may be void of the sensate composition and/or the surface softening composition and/or the lotion composition and vice versa so that when a user contacts the user's skin with the fibrous structure, only the lotion composition contacts the user's skin. A similar case is true for embossed fibrous structures where the embossed areas may, directly and/or indirectly, comprise the sensate composition and/or the surface softening composition and/or the lotion composition and the non-embossed areas may be void of the sensate composition and/or the surface softening composition and/or the lotion composition and vice versa.

The user contacting surface may be present on the fibrous structure and/or sanitary tissue product, for example toilet tissue before use by the user and/or the user contacting surface may be created/formed prior to and/or during use of the fibrous structure and/or sanitary tissue product, for example toilet tissue by the user, such as upon the user applying pressure to the fibrous structure and/or sanitary tissue product, for example toilet tissue as the user contacts the user's skin with the fibrous structure and/or sanitary tissue product, for example toilet tissue.

“Non-sublimable TRPM8 Receptor Triggering Agent” as used herein means a TRPM8 receptor triggering agent that is not capable of subliming and/or does not sublime at a temperature of from about 18° C. to about 80° C. Non-limiting examples of TRPM8 receptor triggering agents that are sublimable and thus do not fall under the “non-sublimable TRPM8 receptor triggering agent” definition include menthol and camphor and WS compounds in general, for example WS23, WS3, and WS5.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Fibrous Structures

The fibrous structures according to the present invention comprise a surface, for example a user contacting surface, comprising one or more TRP receptor triggering agents (“sensates”), for example one or more TRPM8 receptor triggering agents, and optionally, one or more TRP receptor blocking agents, such as TRP receptor blocking agents that block one or more of the following TRP receptors: TRPA1, TRPV, TRPV1, TRPA1V1, TRPV2, TRPV3, and TRPV4, for example TRPA1, TRPV1, and TRPV3. A TRP receptor blocking agent block(s) (for purposes of the present invention, “blocks” includes in addition to 100% or substantially 100% block, dampens, for example dampens to an extent that a sensation experienced by a consumer from a TRP receptor is not unpleasant and/or undesirable rather than provoking a biological response itself) by binding to a TRP receptor.

In one example, the fibrous structure according to the present invention comprises a surface, for example a user contacting surface, comprising a sensate composition, for example a surface softening composition, comprising a surface softening agent and one or more TRP receptor triggering agents (“sensates”), for example one or more TRPM8 receptor triggering agents, and optionally, one or more TRP receptor blocking agents, such as one or more TRP receptor blocking agents that blocks one or more of the following TRP receptors: TRPA1, TRPV, TRPV1, TRPA1V1, TRPV2, TRPV3, and TRPV4, for example TRPA1, TRPV1, and TRPV3.

In another example, the surface of the fibrous structure may comprise a layer of a sensate composition and/or a surface softening composition according to the present invention and a layer of a different sensate composition and/or surface softening composition and/or a lotion composition. The layers of the sensate compositions and/or surface softening compositions and/or lotion composition may be phase registered with one another. In another example, the different sensate compositions and/or surface softening compositions and/or lotion composition may cover different regions of the surface of the fibrous structure, for example they may be in a striped or patterned configuration.

In still another example, the sensate composition and/or surface softening composition of the present invention may cover about 100% and/or greater than 98% and/or greater than 95% and/or greater than 90% of the surface area of the surface, for example the user contacting surface of the fibrous structure.

The sensate composition and/or surface softening composition and/or lotion composition may be applied to a surface, for example a user contacting surface of the fibrous structure by any suitable means known in the art. Any contact or contact-free application suitable for applying the sensate composition and/or surface softening composition and/or lotion composition, such as spraying, dipping, padding, printing, slot extruding, such as in rows or patterns, rotogravure printing, flexographic printing, offset printing, screen printing, mask or stencil application process, and mixtures thereof can be used to apply the sensate composition and/or surface softening composition and/or lotion composition to the surface of the fibrous structure and/or sanitary tissue product, for example toilet tissue. Sensate compositions and/or Surface softening compositions can be applied to the fibrous structure and/or sanitary tissue product before, concurrently, or after the lotion composition application to the fibrous structure and/or sanitary tissue product.

In one example, the sensate composition and/or the surface softening composition and/or the lotion composition is applied to the surface of the fibrous structure during the fibrous structure making process, such as before and/or after drying the fibrous structure.

In another example, the sensate composition and/or the surface softening composition and/or the lotion composition is applied to the surface of the fibrous structure during the converting process.

In another example, the sensate composition may be applied to the surface of the fibrous structure, in the absence of surface softening composition and/or lotion composition, after dissolving into a solvent.

In yet another example, the sensate composition and/or the surface softening composition is applied to the surface of a fibrous structure prior to application of the lotion composition.

The sensate composition and/or the surface softening composition and/or lotion composition can be applied during papermaking and/or converting, especially if applied to the outside layer of a layered fibrous structure and/or sanitary tissue product comprising such layered fibrous structure.

The sensate composition and/or surface softening composition and/or lotion composition can be applied by separate devices or by a single device that has two or more chambers capable of separately delivering the different compositions, especially incompatible, different compositions, such as the sensate composition and/or the surface softening composition and the lotion composition.

The application devices may be sequentially arranged along the papermaking (fibrous structure making) and/or converting process.

The fibrous structure may comprise one or more fibrous structure plies within a single- or multi-ply sanitary tissue product.

### TRP Receptor Triggering Agents

As described above, the TRP receptor triggering agents (“sensates”), for example the TRPM8 receptor triggering agents, of the present invention trigger (“activate”) a TRP receptor, for example a TRPM8 receptor, by a TRP receptor triggering agent (“sensate”), for example a TRPM8 receptor triggering agent, binding to the TRP receptor, for example binding to the TRPM8 receptor, to produce a biological response, in other words to elicit a sensation, such as a cooling sensation in the case of the TRPM8 receptor, that the consumer experiences.

In one example, the TRP receptor triggering agents (“sensates”) comprise one or more TRPM8 receptor triggering agents.

### TRPM8 Receptor Triggering Agents

The fibrous structures of the present invention comprise one or more TRPM8 receptor triggering agents alone and/or in a sensate composition with or without other TRPM8 receptor triggering agents and/or TRP receptor blocking agents and/or other materials.

The TRPM8 receptor triggering agents, may be delivered to a user's skin in a form capable of stimulating the TRPM8 receptor immediately (i.e., within 30 seconds, alternatively 15 seconds) subsequent to contact and/or in a delayed fashion after contact (after 1 minute and/or after 3 minutes or longer). Further, the cooling sensation resulting from stimulating the TRPM8 receptor may be of short duration, for example less than 1 minute and/or less than 45 seconds and/or less than 30 seconds and/or less than 15 seconds and/or less than 10 seconds) and/or may be of longer duration, for example greater than 2 minutes and/or greater than 5 minutes and/or greater than 10 minutes.

In one example, one or more TRPM8 receptor triggering agents may be present on the fibrous structure and/or in a sensate composition present on a fibrous structure at a level of greater than 0% to less than about 3% and/or greater than 0% to less than about 2% and/or greater than 0% to less than about 1.5% and/or greater than 0% to less than about 1% and/or greater than 0% to less than about 0.5% and/or from about 0.01% to less than about 2% and/or from about 0.01% to less than about 1% by weight of the fibrous structure and/or sensate composition, respectively.

In one example, no TRPM8 receptor triggering agent is present on the fibrous structure and/or in a sensate composition present on a fibrous structure at a level of greater than 3% and/or greater than 2% and/or greater than 1% greater than 0.5% by weight of the fibrous structure and/or sensate composition, respectively.

In one example, at least one of the TRPM8 receptor triggering agents is transferable to a surface, such as skin, for example a user's skin, such as a user's anal skin.

During use of the fibrous structure of the present invention by a user, at least one of the TRPM8 receptor triggering agents is in contact with a surface, for example a user's skin, such as a user's anal skin.

In one example, a fibrous structure of the present invention comprising a sensate composition comprising one or more TRPM8 receptor triggering agents and optionally, at least one TRP receptor blocking agent, elicits a response from a TRPM8 receptor within 30 seconds and/or 15 seconds and/or 5 seconds and/or 1 second of contact with a surface, for example with a user's skin, such as a user's anal skin.

In one example, a fibrous structure of the present invention comprising a sensate composition comprising one or more TRPM8 receptor triggering agents and optionally, at least one TRP receptor blocking agent, elicits a response from a TRPM8 receptor that exhibits a duration of less than 1 minute and/or less than 45 seconds and/or less than 30 seconds and/or less than 15 seconds and/or less than 10 seconds.

In one example, one or more of the TRPM8 receptor triggering agents are present in a sensate composition, such as a surface softening composition, for example a surface softening composition, at a level of at least 0.25% by weight of the sensate composition.

TRPM8 receptor triggering agents triggering agents trigger a TRPM8 receptor eliciting a cooling sensation. Non-limiting examples of suitable TRPM8 receptor triggering agents that elicit a cooling sensation are described in US Published Patent Applications US2004/0081680A1, US2004/0082654 A1, US 2004/0067970 A1, and US 2005/0049553 A1, and U.S. Pat. Nos. 4,296,255; 4,296,093; 4,226,988; 4,193,936; 4,178,459; 4,153,679; 4,150,052; 4,070,496; 4,070,449; 4,034,109; 4,033,994; 4,032,661; 4,020,153; 4,459,425; 6,267,974; 6,592,884; 6,328,982; 6,359,168; 6,214,788; 5,608,119; and 6,884,906 all of which are incorporated herein by reference. In one example, the TRPM8 receptor triggering agent is selected from the TRPM8 receptor triggering agents described in US 2004/0082654 A1.

The TRPM8 receptor triggering agents can be present on a surface of a fibrous structure, for example on a user contacting surface of the fibrous structure and/or present in a sensate composition (present on a surface of a fibrous structure), for example a surface softening composition alone or in combination with other TRPM8 receptor triggering agents, for example ketals such as Frescolat® MGA (available from Symrise), cyclic carboxamides such as WS-3 (available as ISE 3000 from Qarôma, Inc.), aliphatic carboxamides such as WS-23 (available as ISE 1000 from Qarôma, Inc.), cyclohexanol derivatives such as Coolact P® (available from Takasago), and cyclohexyl derivatives such as TK10 (Takasago) and menthyl lactate, also known as Frescolat® ML (Symrise), and optionally, one or more TRP receptor blocking agents and/or other materials, which are present on a surface of the fibrous structure, for example present on a user contacting surface of the fibrous structure.

In one example, the TRPM8 receptor triggering agent is selected from the group consisting of: carboxamides, ketals, esters, ethers, alcohols, and mixtures thereof.

In one example, the TRPM8 receptor triggering agent comprises a carboxamide, for example a ρ-menthanecarboxamide, such as a ρ-menthanecarboxamide selected from the group consisting of: N-ethyl-ρ-menthan-3-carboxamide, N-ethoxycarbonylmethyl-ρ-menthan-3-carboxamide, [N-(4-methoxyphenyl)-ρ-menthan-3-carboxamide], N-tert-butyl-ρ-menthan-3-carboxamide, N-(4-cyanomethy-phenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide, and mixtures thereof.

In another example, the TRPM8 receptor triggering agent carboxamide is N,2,3-trimethyl-2-isopropylbutanamide.

In another example, the TRPM8 receptor triggering agent carboxamide is selected from the group consisting of: cyclic carboxamides, aliphatic carboxamides, and mixtures thereof.

In yet another example, the TRPM8 receptor triggering agent comprises a ketal.

In still another example, the TRPM8 receptor triggering agent comprises an ester, for example a menthane carboxy ester, such as a menthane carboxy ester selected from the group consisting of: 1-glyceryl-ρ-menthane-3-carboxylate, ethylene glycol ρ-menthane-3-carboxylate, and mixtures thereof.

In another example, the TRPM8 receptor triggering agent ester is a menthyl ester, for example a menthyl ester selected from the group consisting of: menthyl acetate, menthyl acetoacetate, menthyl lactate, monomenthyl succinate, and mixtures thereof.

In yet another example, the TPRM8 receptor triggering agent ester is selected from the group consisting of: N-((5-methyl-2-(1-methylethyl)cyclohexyl)carbonyl)glycine ethyl ester, N-((5-methyl-2-(1-methylethyl)cyclohexyl)carbonyl)alanine ethyl ester, and mixtures thereof.

In another example, the TRPM8 receptor triggering agent comprises an ether.

In another example, the TRPM8 receptor triggering agent comprises an alcohol, for example an alcohol selected from the group consisting of: 3-(1-menthoxy)-propane-1,2-diol, isopulegol, ρ-menthane-3,8-diol, and mixtures thereof.

In yet another example, the TRPM8 receptor triggering agent comprises a cyclohexanol moiety.

In even yet another example, the TRPM8 receptor triggering agent comprises a menthol derivative.

In even yet another example, the TRPM8 receptor triggering agent comprises a cyclohexyl moiety.

In another example, the TRPM8 receptor triggering agent comprises an alpha-keto enamine derivative, for example an alpha-keto enamine derivative selected from the group consisting of: 3-methyl-2-(1-pyrrolidinyl)-2-cyclopenten-1-one, 5-methyl-2-(1-pyrrolidinyl)-2-cyclopenten-1-one, 2,5-dimethyl-4-(1-pyrrolidinyl)-3(2H)-furanone, and mixtures thereof.

In yet another example, the TRPM8 receptor triggering agent is 1-[2-hydroxyphenyl]-4-[2-nitrophenyl]-1,2,3,6-tetrahydropyrimidine-2-one.

In another example, the TRPM8 receptor triggering agent is selected from the group consisting of: N-substituted menthanecarboxamide, 1-(2-hydroxyphenyl-)-4-(2-nitrophenyl-)-1,2,3,6-tetrahydropyrimidine-2-one and derivatives thereof, ethyl 3-(ρ-menthane-3-carboxamido) acetate, N-(4-methoxyphenyl)-ρ-menthane-3-carboxamide, N-t-butyl-ρ-menthane-3-carboxamide, 1-glyceryl-ρ-menthane-3-carboxylate, ethylene glycol ρ-menthane-3-carboxylate, 3-(1-menthoxy)-2-methylpropane-1,2-diol, geraniol, eucalyptol, linaool, hydroxycitronellal, paramenthane-3,8-diol, menthyl pyrrolidone carboxlate, 2-isopropyl-5-methylcyclohexyl-4-(dimethylamino)-4-oxobutanoate, (−)-Cubebol, N-(4-cyanomenthylphenyl)-p-menthanecarboxamide, (1R,3R,4S)-3-menthyl-3,6-dioxaheptanoate, (1R,2S,5R)-3-menthyl-methoxy acetate, (1R,2S,5R)-3-menthyl-3,6,9-trioxadecanoate, (1R,2S,5R)-3-menthyl-(2-hydroxyethoxy) acetate, (1R,2S,5R)-menthyl-11-hydroxy-3,6,9-trioxaundacanoate, (−)-Isopulegol, cis ρ-menthane-3,8-diol, trans ρ-menthane-3,8-diol, 3-(1-menthoxy) propane-1,2-diol, menthol and derivatives thereof, menthyl lactate, methyl salicylate, and mixtures thereof.

In another example, the TRPM8 receptor triggering agent is selected from the group consisting of: esters of menthol and menthol derivatives, menthylcarboxylic acid ester derivatives, carboxamides, cyclohexane carboxy ester derivatives, cyclohexylmethyl carboxylate derivatives, cyclohexyl anthranilate derivatives, omega-amino cyclohexyl carboxylate derivatives, omega-amino cyclohexane carboxy ester derivatives, omega-amino cyclohexylmethyl carboxylate derivatives, and mixtures thereof.

Non-limiting examples of suitable TRPM8 receptor triggering agents that provide a cooling sensation that can be provided alone or in combination include 1-(2-hydroxyphenyl-)-4-(2-nitrophenyl-)-1,2,3,6-tetrahydropyrimidine-2-one, also known as Icilin available from Biomol International, and derivatives thereof; ethyl 3-(ρ-menthane-3-carboxamido) acetate, also known as WS-5 (available from Millenium Specialty Chemicals as WinSense™ Extra); N-(4-methoxyphenyl)-p-menthane-3-carboxamide, also known as WS-12; N-t-butyl-p-menthane-3-carboxamide, also known as WS-14; 1-glyceryl-p-menthane-3-carboxylate, also known as WS-30; ethylene glycol p-menthane-3-carboxylate, also known as WS-4; 3-(1-menthoxy)-2-methylpropane-1,2-diol; geraniol; eucalyptol; linaool; hydroxycitronellal; paramenthane-3,8-diol, also known as PMD-38 (Takasago International); menthyl pyrrolidone carboxlate, also known as Questice® (available from Quest International); 2-isopropyl-5-methylcyclohexyl-4-(dimethylamino)-4-oxobutanoate; (−)-Cubebol (also known as (−)-(1R,4S,5R,6R,7S,10R)-7-isopropyl-4,10-dimethyl-tricyclo[4.4.0.0(1,5)]decan-4-ol); N-(4-cyanomenthylphenyl)-p-menthanecarboxamide; (1R,3R,4S)-3-menthyl-3,6-dioxaheptanoate; (1R,2S,5R)-3-menthyl-methoxy acetate; (1R,2S,5R)-3-menthyl-3,6,9-trioxadecanoate; (1R,2S,5R)-3-menthyl-(2-hydroxyethoxy) acetate; (1R,2S,5R)-menthyl-11-hydroxy-3,6,9-trioxaundacanoate; (−)-Isopulegol; cis and trans ρ-menthane-3,8-diol, also known as Coolact 38D® (Takasago International); and 3-(1-Menthoxy) propane-1,2-diol (Cooling Agent 10) from Taksago International menthol and derivatives.

In one example, the fibrous structures according to the present invention and/or sensate composition present on a surface of the fibrous structure may comprise at least one TRPM8 receptor triggering agent selected from the group consisting of: menthol, WS-3, WS-23, methyl salicylate, WS-5, menthone glycerin acetal (Frescolat® MGA), MML, menthyl lactate, WinSenseXtra 500 (WS3, WS-23, Menthyl Lactate), N-substituted menthanecarboxamide, 1-(2-hydroxyphenyl-)-4-(2-nitrophenyl-)-1,2,3,6-tetrahydropyrimidine-2-one and derivatives thereof, ethyl 3-(p-menthane-3-carboxamido) acetate, N-(4-methoxyphenyl)-p-menthane-3-carboxamide, 1-glyceryl-p-menthane-3-carboxylate, ethylene glycol p-menthane-3-carboxylate, 3-(1-menthoxy)-2-methylpropane-1,2-diol, geraniol, eucalyptol, linaool, hydroxycitronellal, paramenthane-3,8-diol, menthyl pyrrolidone carboxlate, 2-isopropyl-5-methylcyclohexyl-4-(dimethylamino)-4-oxobutanoate, (−)-Cubebol; N-(4-cyanomenthylphenyl)-p-menthanecarboxamide, (1R,3R,4S)-3-menthyl-3,6-dioxaheptanoate, (1R,2S,5R)-3-menthyl-methoxy acetate, (1R,2S,5R)-3-menthyl-3,6,9-trioxadecanoate, (1R,2S,5R)-3-menthyl-(2-hydroxyethoxy) acetate, (1R,2S,5R)-menthyl-11-hydroxy-3,6,9-trioxaundacanoate, (−)-Isopulegol; cis and trans ρ-menthane-3,8-diol, and Cooling Agent 10 and derivatives, and mixtures thereof.

The TRPM8 receptor triggering agents may be of natural or synthetic origin. The most well-known TRPM8 receptor triggering agent is menthol, particularly 1-menthol, which is found naturally in peppermint oil, notably of Mentha arvensis L and Mentha viridis L. Of the menthol isomers, the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces. Other isomers of menthol (neomenthol, isomenthol and neoisomenthol) have somewhat similar, but not identical odor and taste, i.e., some having disagreeable notes described as earthy, camphor, musty. The principal difference among the isomers is in their cooling potency. L-menthol provides the most potent cooling, i.e., having the lowest cooling threshold of about 800 ppb, i.e., the concentration where the cooling effect could be clearly recognized. At this level, there is no cooling effect for the other isomers. For example, d-neomenthol is reported to have a cooling threshold of about 25,000 ppb and 1-neomenthol about 3,000 ppb. (R. Emberger and R. Hopp, “Synthesis and Sensory Characterization of Menthol Enantiomers and Their Derivatives for the Use in Nature Identical Peppermint Oils,” Specialty Chemicals (1987), 7(3), 193-201). This study demonstrated the outstanding sensory properties of 1-menthol in terms of cooling and freshness and the influence of stereochemistry on the activity of these molecules.

Among synthetic TRPM8 receptor triggering agents, many are derivatives of or are structurally related to menthol, i.e., containing the cyclohexane moiety, and derivatized with functional groups including carboxamide, ketal, ester, ether and alcohol. Examples include the ρ-menthanecarboxamide compounds, such as N-ethyl-ρ-menthan-3-carboxamide, known commercially as “WS-3”, and others in the series, such as WS-5 (N-ethoxycarbonylmethyl-ρ-menthan-3-carboxamide), WS-12 [N-(4-methoxyphenyl)-ρ-menthan-3-carboxamide] and WS-14 (N-tert-butyl-ρ-menthan-3-carboxamide). Examples of menthane carboxy esters include WS-4 and WS-30. An example of a synthetic carboxamide TRPM8 receptor triggering agent that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and ρ-menthane-3,8-diol (under the tradename Coolact 38D); menthone glycerol acetal known as MGA; menthyl esters such as menthyl acetate, menthyl acetoacetate, menthyl lactate known as Frescolat® ML supplied by Haarmann and Reimer, and monomenthyl succinate under the tradename Physcool from V. Mane. TK-10 is described in U.S. Pat. No. 4,459,425. Other alcohol and ether derivatives of menthol are described e.g., in GB 1,315,626 and in U.S. Pat. Nos. 4,029,759; 5,608,119; and 6,956,139. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide. Other N-substituted ρ-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N-((5-methyl-2-(1-methylethyl)cyclohexyl)carbonyl)alanine ethyl ester. Menthyl esters, including those of amino acids such as glycine and alanine are disclosed e.g., in EP 310 299 and in U.S. Pat. Nos. 3,111,127; 3,917,613; 3,991,178; 5,703,123; 5,725,865; 5,843,466; 6,365,215; 6,451,844; and 6,884,903. Ketal derivatives are described, e.g., in U.S. Pat. Nos. 5,266,592; 5,977,166 and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200, R. Eccles, J. Pharm. Pharmacol., (1994), 46, 618-630, and J. C. Leffingwell, Leffingwell & Assoc. http://leffingwell.com/cooler_than_menthol.htm (2016).

Molecules with chiral centers can drive different biological responses depending upon the spatial orientation of specific moieties on those molecules. The biological responses tend to differ where these molecules interact with a receptor. In the flavor and fragrance realm, a well-known example of such chiral diversity is Carvone. The R-(−) enantiomers of Carvone connotes a spearmint taste and scent, where the S-(+) enantiomer has a taste and smell like caraway seeds. Limonene is another molecule where the spatial orientation of the chiral center affects its scent. For example, the R-(+) isomer of limonene has a citrus scent, where the S-(−) isomer smells like turpentine. For synthetic molecules, the ability to control the stereochemistry during the synthesis steps, gives the ability to select for the finished molecule with the desired sensorial properties. The object of this invention is a method of synthesis to control the stereochemistry of amino acid substituted aryl cyclohexane carboxamides.

In one example, non-limiting examples of TRPM8 receptor triggering agents include esters of menthol and menthol derivatives, menthylcarboxylic acid ester derivatives, carboxamides, cyclohexane carboxy ester derivatives, cyclohexylmethyl carboxylate derivatives, cyclohexyl anthranilate derivatives, omega-amino cyclohexyl carboxylate derivatives, omega-amino cyclohexane carboxy ester derivatives, and omega-amino cyclohexylmethyl carboxylate derivatives.

In another example, the TRPM8 receptor triggering agents are void or substantially void of esters of menthol and menthol derivatives, menthylcarboxylic acid ester derivatives, carboxamides, cyclohexane carboxy ester derivatives, cyclohexylmethyl carboxylate derivatives, cyclohexyl anthranilate derivatives, omega-amino cyclohexyl carboxylate derivatives, omega-amino cyclohexane carboxy ester derivatives, omega-amino cyclohexylmethyl carboxylate derivatives, and camphor since these TRPM8 receptor triggering agents tend to elicit too long of a sensation making the user's experience unacceptable, unpleasant, and/or undesirable.

In another example, the TRPM8 receptor triggering agents are void of menthol and camphor.

In yet another example, the TRPM8 receptor triggering agents comprise non-sublimable TRPM8 receptor triggering agents.

In still another example, the TRPM8 receptor triggering agents comprise TRPM8 triggering agents that do not create an olfactory response.

In one example, the TRPM8 receptor triggering agents present, neat or in a sensate composition, on a user contacting surface of the fibrous structure of the present invention, do not function to bind the fibers and/or filaments of the fibrous structure.

In one example, the sensate composition is void of TRPM8 receptor triggering agents selected from the group consisting of: esters of menthol and menthol derivatives, menthylcarboxylic acid ester derivatives, carboxamides, cyclohexane carboxy ester derivatives, cyclohexylmethyl carboxylate derivatives, cyclohexyl anthranilate derivatives, omega-amino cyclohexyl carboxylate derivatives, omega-amino cyclohexane carboxy ester derivatives, omega-amino cyclohexylmethyl carboxylate derivatives, camphor, and mixtures thereof.

In another example, the sensate composition is void of TRPM8 receptor triggering agents selected from the group consisting of: menthol, camphor, and mixtures thereof.

### TRP Receptor Blocking Agents

In addition to the one or more TRPM8 receptor triggering agents, the fibrous structure and/or sensate composition present on the fibrous structure may further comprise one or more TRP receptor blocking agents that blocks one or more and/or two or more TRP receptors, for example one or more and/or two or more of the TRPA1, TRPV, TRPV1, TRPA1V1, TRPV2, TRPV3, and TRPV4 receptors. In one example, at least one of the TRP receptor blocking agents is a selective blocking agent. In another example, at least one of the TRP receptor blocking agents is a reversible blocking agent.

As described above, a TRP receptor blocking agent blocks (for purposes of the present invention, “blocks” includes, in addition to 100% blocked or substantially 100% blocked, dampens, for example dampens to an extent that a sensation experienced by a consumer from a TRP receptor is not unpleasant and/or undesirable rather than provoking a biological response itself) by binding to a TRP receptor.

Certain TRP receptor blocking agents may block more than one TRP receptor while others may block a specific TRP receptor. Non-limiting examples of TRP receptor blocking agents and the TRP receptors that they block are described below.

When present, the one or more TRP receptor blocking agents is present on the fibrous structure and/or in the sensate composition at a level of greater than 0% to about 0.5% by weight of the fibrous structure or sensate composition, respectively. In one example, the one or more TRP receptor blocking agents is present on the fibrous structure and/or in the sensate composition at a level of from about 100 ppm to about 2000 ppm and/or at a level of from about 200 ppm to about 1000 ppm.

In one example, one or more TRP receptor blocking agents block two or more and/or three or more and/or four or more and/or five or more and/or all of the TRPA1, TRPV, TRPV1, TRPA1V1, TRPV2, TRPV3, and TRPV4 receptors.

a. TRPA1, TRPV1, TRPA1V1, TRPV3 Receptor Blocking Agents

Non-limiting examples of blocking agents that block the TRPA1, TRPV1, TRPA1V1, and TRPV3 receptors include curcumin, manganese gluconate, manganese citrate, magnesium sulfate, tyramine, DL-lactic acid, and L-(+)-lactic acid.

b. TRPA1, TRPV1, TRPA1V1 Receptor Blocking Agents

Non-limiting examples of blocking agents that block the TRPA1, TRPV1, and TRPA1V1 receptors include 2-hexyldecanoic acid and 2-naphthyl anthranilate.

c. TRPA1, TRPV1, TRPV3 Receptor Blocking Agents

Non-limiting examples of blocking agents that block the TRPA1, TRPV1, and TRPV3 receptors include β-cyclodextrin, 3-octyl acetate, phenoxyethyl isobutyrate, 6-methyl-5-hepten-2-one, farnesol, n-propyl gallate, laureth 6, laureth 9, nordihydroguaiaretic acid, madecassic acid, asiatic acid, 4-hexyl resorcinol, Aescin IB, Aescin IA, d-platycodin, trans-garcinolic acid, agaric acid, celastrol, and polyethyleneimine (“PEI”), for example PEIs that exhibit molecular weights of about 2000 g/mol (PEI 2000).

d. TRPA1, TRPV1 Receptor Blocking Agents

Non-limiting examples of blocking agents that block the TRPA1 and TRPV1 receptors include α-amylcinnamaldehyde dimethyl acetal, benzaldehyde glyceryl acetal, apritone, potassium gluconate, d-gluconic acid sodium salt, butyl isobutyrate, 1-(1,2,3,4,5,6,7,8-octahydro-2,3,8,8,-tetramethyl-2-naphthyl)ethan-1-one, and myrtenol.

e. TRPA1, TRPA1V1 Receptor Blocking Agents

Non-limiting examples of blocking agents that block the TRPA1 and TRPA1V1 receptors include Z-methyl epi jasmonate, cis-4-decen-1-ol, isobornyl isobutyrate, 4-acetoxy-2,5-dimethyl-3(2H)furanone, 3,3,5-trimethyl cylcohexanol, lactamide monoethanolamine, bornyl valerate, 4-methyl-2-phenyl-2-pentenal, phenyl salicylate, and difurfuryldisulfide.

f. TRPA1, TRPV3 Receptor Blocking Agents

Non-limiting examples of blocking agents that block the TRPA1 and TRPV3 receptors include 6-hydroxydihydrotheaspirane, tripal, (+)-sodium L-ascorbate, and ethylene brassylate.

g. TRPV1, TRPA1V1 Receptor Blocking Agents

Non-limiting examples of blocking agents that block the TRPV1 and TRPA1V1 receptors include octyl acetate, 4-(4-hydroxy-phenyl)-butan-2-one, 2-cyclohexylethyl acetate, 1-methylpyrrole-2-carboxaldehyde, and δ-hexalactone.

h. TRPV1, TRPV3 Receptor Blocking Agents

Non-limiting examples of blocking agents that block the TRPV1 and TRPV3 receptors include cinnamon bark oil and (−)-carvyl acetate.

i. TRPA1V1, TRPV3 Receptor Blocking Agents

A non-limiting example of a blocking agent that blocks the TRPA1V1 and TRPV3 receptors includes L-(+)-tartaric acid.

j. TRPA1 Receptor Blocking Agents

Non-limiting examples of blocking agents that block the TRPA1 receptor (“TRPA1 receptor blocking agent”) include menthalactone, propanoic acid, isopropyl cinnamate, furfuryl mercaptan, 5-methylquinoxaline, dimethylphenethylcarinol acetate, 2-methylbenzenethiol, maltol isobutyrate, p-mentha-8-thiol-3-one, oleyl alcohol, 1,1-dimethoxy-dodecane, methyl 4-pentenoate, 2,2,4-trimethyl-1,3,dioxolane, 2-methylphenol, ethyl 2-ethylbutyrate, 6,10-dimethyl-9-undecen-2-one, tetrahydro-2-(3-phenylpropyl)-furan, β-terpineol, 3-heptyldihydro-5-methylfuran-2(3H)-one, hexyl-2-methylbutyrate, rosoxide, (s)-verbenone, biphenyl, 2-ethylbutyl acetate, cis-5-octen-1-ol, 2-(2-octenyl)-cyclopentanone, 4-methyl-2-pentyl-1,3-dioxolane, isobornyl isovalerate, γ-dodecalactone, 2,4-dimethylanisole, (+−)-1-phenylethanol, 2-ethyl-3-hydroxy-4H-pyran-4-one, 3-mercapto-2-butanol, ethyl trans-2-hexanoate, diallyl sulfide, methyl 3-(methylthio)propionate, methyl laurate, 2-methyl-1,3,-dithiolane, ethyl-3-phenylglycidate, manganese chloride tetrahydrate, allyl caprylate, diphenyl disulfide, δ-tocopherol, 9-cycloheptadecen-1-one, tannic acid, fenchyl alcohol, raspberry ketone, 4-(4-hydroxyphenyl)butan-2-one, 2-(1,3-dimethyl-2,6-dioxo-1,2,3,6-tetrahydro-7H-purin-7-yl)-N-[4-(1-methylpropyl)phenyl]acetamide, 2-(1,3-dimethyl-2,6-dioxo-1,2,3,6-tetrahydro-7H-purin-7-yl)-N-(4-isopropyl)phenyl)acetamide, (1E,3E)-1-(4-fluorophenyl)-2-methyl-1-pentene-3-one oxime, 4-(4-chlorophenyl)-3-methyl-3-buten-2-one oxime, and isoamyl acetate.

In one example, the TRPA1 receptor blocking agent is a selective TRPA1 receptor blocking agent, for example the selective TRPA1 receptor blocking agent is selected from the group consisting of: raspberry ketone, 4-(4-hydroxyphenyl)butan-2-one, 2-(1,3-dimethyl-2,6-dioxo-1,2,3,6-tetrahydro-7H-purin-7-yl)-N-[4-(1-methylpropyl)phenyl]acetamide, 2-(1,3-dimethyl-2,6-dioxo-1,2,3,6-tetrahydro-7H-purin-7-yl)-N-(4-isopropyl)phenyl)acetamide, (1E,3E)-1-(4-fluorophenyl)-2-methyl-1-pentene-3-one oxime, and mixtures thereof.

In one example, the TRPA1 receptor blocking agent is a reversible TRPA1 receptor blocking agent, for example 4-(4-chlorophenyl)-3-methyl-3-buten-2-one oxime.

k. TRPV1 Receptor Blocking Agents

Non-limiting examples of blocking agents that block the TRPV1 receptor (“TRPV1 receptor blocking agent”) include 3-nonen-2-one, 2-octenoic acid, α-lonol, β-lonol, 2-methoxycinnamaldehyde, 4-(4-methoxyphenyl)-2-butanone, N,N-dimethylanthranilic acid methyl ester, methyl 4-phenylbutyrate, decyl acetate, ethyl 4-pentenoate, trans-2-hexenyl hexanoate, palmitic acid, phytol acetate, ethyl 5-oxodecanoate, n-propylparaben, isobutyl N-methylanthranilate, sandalwood oil, celery, chicory, styrallyl butyrate, rhodinyl acetate, 2-methylbutyl isovalerate, 3-butylidenephthalide, piperitone, isosafrole, geraniol, β-bisabolene, luteolin, N-oleoylethanolamine, trans-2-cis-6-nonadienal, isoborneol, 5-methyl-2-phenyl-2-hexenal, 2-tridecanone, allyl α-ionone, benzyl benzoate, isobutyl hexanoate, laevo menthyl acetate, methyl anthranilate, 3,6-dihydro-3′-(trifluomethyl)-N-[4-[(trifluoromethyl)sulfonyl]phenyl]-[1(2H),2′-bipyridine]-4-carboxamide, 2-[(2E)-3,7-dimethylocta-2,6-dien-1-yl]cyclopentan-1-one, 3-amino-5-[[2-[(2-methyoxyethyl)amino]-6-[4-(trifluoromethyl)phenyl]-4-pyrimidinyl]oxy]-2(1H)-quinoxalinone, (2E)-N-(2,3-dihydro-1,4-benzodioxin-6-yl)-3-[4-(1,1-dimethylethyl)phenyl]-2-propenamide, 4-(3-chloro-2-pyridinyl)-N-[4-(1,1-dimethylethyl)phenyl]-1-piperazinecarboxamide, and magnesium sulfate.

In one example, the following TRPV1 receptor blocking agents are considered potent and selective: 3,6-dihydro-3′-(trifluomethyl)-N-[4-[(trifluoromethyl)sulfonyl]phenyl]-[1(2H),2′-bipyridine]-4-carboxamide, 2-[(2E)-3,7-dimethylocta-2,6-dien-1-yl]cyclopentan-1-one, 3-amino-5-[[2-[(2-methyoxyethyl)amino]-6-[4-(trifluoromethyl)phenyl]-4-pyrimidinyl]oxy]-2 (1H)-quinoxalinone, (2E)-N-(2,3-dihydro-1,4-benzodioxin-6-yl)-3-[4-(1,1-dimethylethyl)phenyl]-2-propenamide, and 4-(3-chloro-2-pyridinyl)-N-[4-(1,1-dimethylethyl)phenyl]-1-piperazinecarboxamide.

l. TRPA1V1 Receptor Blocking Agents

Non-limiting examples of blocking agents that block the TRPA1V1 receptor (“TRPA1V1 receptor blocking agent”) include 2,5-dimethyl-1,4-dithiane-2,5-diol, methyl phenyl disulfide, α-α-dimethylbenzene propanol, propyl isobutyrate, methyl jasmonate, γ-methyl-γ-decanolactone, pyruvic acid cis-3-hexen-1-yl ester, d-vebenol, 6-acetoxydihydrotheaspirane, hexyl isothiocyanate, citric extract, castor oil, nerol, nicotinic acid, sodium dodecyl sulfate, 4-anisyl acetate, 4-thujanol, 2-methyl piperidine, oleoresin ginger, propenylguaethol, propyl mercaptan, 2-methylacetophenone, irone, ethyl 2-octenoate, DBE-5 dibasic ester, glycerol oleate, β-methyl-γ-octalactone, 1-isopropyl-2-methoxy-4-methylbenzene, allyl phenylacetate, bornyl acetate, methyl eugenol ether, wintergreen oil, vanillin acetate, and sodium phosphate dibasic.

m. TRPV3 Receptor Blocking Agents

Non-limiting examples of blocking agents that block the TRPV3 receptor (“TRPV3 receptor blocking agent”) include disodium phosphate, dipotassium phosphate, sodium bicarbonate, trans-2-decenal, (−)-α-bisabolol, 4-allyl-2,6-dimethoxyphenol, farnesal, capsaicin, 2′,4′-dimethylacetophenone, 2-methoxy-4-vinylphenol, β-ionone, carrot oil, perillyl alcohol, bisabolol, methyl linolenate, 3,5,5-trimethylcyclohexane-1,2-dione, fenugreek, α-monoolein, camphor white oil, (+/−)-4-heptanolide, cyclal c, and octyl 2-furoate.

n. TRPV Receptor Blocking Agents

Non-limiting examples of blocking agents that block the TRPV receptor (“TRPV receptor blocking agent”) include ammoniated ruthenium oxychloride and 6-iodonordihydrocapsaicin. These TRPV receptor blocking agents are considered nonselective.

o. TRPV2 Receptor Blocking Agents

A non-limiting example of a blocking agent that blocks the TRPV2 receptor (“TRPV2 receptor blocking agent”) includes 2-[[3-(3,4-dimethoxyphenyl)-1-oxo-2-propenyl]amino]benzoic acid.

p. TRPV4 Receptor Blocking Agents

Non-limiting examples of blocking agents that block the TRPV4 receptor (“TRPV4 receptor blocking agent”) include 3-([1,4′-bipiperidin]-1′-ylmethyl)-7-bromo-N-(1-phenylcyclopropyl)-2-[3-(trifluoromethyl)phenyl]-4-quinolinecarboxamide, 2-methyl-1-[3-(4-morpholinyl)propyl]-5-phenyl-N-[3-(trifluoromethyl)phenyl]-1H-pyrrole-3-carboxamide, which are both considered potent and selective, and 2,4-Dichloro-N-isopropyl-N-(2-isopropylaminoethyl)benzenesulfonamide, which is considered selective.

In another example, non-limiting example of blocking agents that block the TRPV4 receptor (“TRPV4 receptor blocking agent”) include 3-([1,4′-bipiperidin]-1′-ylmethyl)-7-bromo-N-(1-phenylcyclopropyl)-2-[3-(trifluoromethyl)phenyl]-4-quinolinecarboxamide, 2-methyl-1-[3-(4-morpholinyl)propyl]-5-phenyl-N-[3-(trifluoromethyl)phenyl]-1H-pyrrole-3-carboxamide, and mixtures thereof.

In yet another example, a blocking agent that blocks the TRPV4 receptor (“TRPV4 receptor blocking agent”) is 2,4-Dichloro-N-isopropyl-N-(2-isopropylaminoethyl)benzenesulfonamide.

In one example, at least one of the TRP receptor blocking agents of the present invention is selected from the group consisting of: apritone, (1E,3E)-1-(4-Fluorophenyl)-2-methyl-1-pentene-3-one oxime, 2-(1,3-Dimethyl-2,6-dioxo-1,2,3,6-tetrahydro-7H-purin-7-yl)-N-(4-isopropylphenyl)acetamide, 2-(1,3-Dimethyl-2,6-dioxo-1,2,3,6-tetrahydro-7H-purin-7-yl)-N-[4-(1-methylpropyl)phenyl] acetamide, 4-(4-Chlorophenyl)-3-methyl-3-buten-2-one oxime, cinnamon bark oil, γ-Dodecalactone, vanillic acid, γ-Menthyl Decalacatone, trans, trans-2,4-Nonadienal, menthyl naphthalenyl ketone or derivative thereof, ammoniated ruthenium oxychloride, 6-Iodonordihyrocapsaicin, 3,6-Dihydro-3′-(trifluoromethyl)-N-[4-[(trifluoromethyl)sulfonyl]phenyl]-[1(2H),2′-bipyridine]-4-carboxamide, 3-Amino-5-[[2-[(2-methoxy ethyl)amino]-6-[4-(trifluoromethyl)phenyl]-4-pyrimidinyl]oxy]-2(1H)-quinoxalinone, (2E)-N-(2,3-Dihydro-1,4-benzodioxin-6-yl)-3-[4-(1,1-dimethylethyl)phenyl]-2-propenamide, 4-(3-Chloro-2-pyridinyl)-N-[4-(1,1-dimethylethyl)phenyl]-1-piperazinecarboxamide, 2-[[3-(3,4-Dimethoxyphenyl)-1-oxo-2-propenyl]amino]benzoic acid, 3-([1,4′-Bipiperidin]-1′-ylmethyl)-7-bromo-N-(1-phenylcyclopropyl)-2-[3-(trifluoromethyl)phenyl]-4-quinolinecarboxamide, 2-Methyl-1-[3-(4-morpholinyl)propyl]-5-phenyl-N-[3-(trifluoromethyl)phenyl]-1H-pyrrole-3-carboxamide, 2,4-Dichloro-N-isopropyl-N-(2-isopropylaminoethyl)benzenesulfonamide, and mixtures thereof.

In another example, at least one of the TRP receptor blocking agents of the present invention is selected from the group consisting of: (1E,3E)-1-(4-Fluorophenyl)-2-methyl-1-pentene-3-one oxime, 2-(1,3-Dimethyl-2,6-dioxo-1,2,3,6-tetrahydro-7H-purin-7-yl)-N-(4-isopropylphenyl)acetamide, 2-(1,3-Dimethyl-2,6-dioxo-1,2,3,6-tetrahydro-7H-purin-7-yl)-N-[4-(1-methylpropyl)phenyl]acetamide, 4-(4-Chlorophenyl)-3-methyl-3-buten-2-one oxime, and mixtures thereof.

In even another example, at least one of the TRP receptor blocking agents of the present invention is selected from the group consisting of: ammoniated ruthenium oxychloride, 6-iodonordihyrocapsaicin, and mixtures thereof.

In still another example, at least one of the TRP receptor blocking agents of the present invention is selected from the group consisting of: 3,6-Dihydro-3′-(trifluoromethyl)-N-[4-[(trifluoromethyl)sulfonyl]phenyl]-[1(2H),2′-bipyridine]-4-carboxamide, 3-Amino-5-[[2-[(2-methoxy ethyl)amino]-6-[4-(trifluoromethyl)phenyl]-4-pyrimidinyl]oxy]-2(1H)-quinoxalinone, (2E)-N-(2,3-Dihydro-1,4-benzodioxin-6-yl)-3-[4-(1,1-dimethylethyl)phenyl]-2-propenamide, 4-(3-Chloro-2-pyridinyl)-N-[4-(1,1-dimethylethyl)phenyl]-1-piperazinecarboxamide, and mixtures thereof.

In another example, at least one of the TRP receptor blocking agents of the present invention comprises 2-[[3-(3,4-Dimethoxyphenyl)-1-oxo-2-propenyl]amino]benzoic acid.

In yet another example, at least one of the TRP receptor blocking agents of the present invention is selected from the group consisting of: 3-([1,4′-Bipiperidin]-1′-ylmethyl)-7-bromo-N-(1-phenylcyclopropyl)-2-[3-(trifluoromethyl)phenyl]-4-quinolinecarboxami de, 2-Methyl-1-[3-(4-morpholinyl)propyl]-5-phenyl-N-[3-(trifluoromethyl)phenyl]-1H-pyrrole-3-carboxamide, 2,4-Dichloro-N-isopropyl-N-(2-isopropylaminoethyl)benzenesulfonamide, and mixtures thereof.

### Sensate Composition

The one or more TRP receptor agents may be present in a sensate composition of the present invention with one or more other materials, for example one or more additives selected from the group consisting of: surface softening agents, humectants, emollients, immobilizing agents, preservatives, and mixtures thereof. In one example, the sensate composition is a surface softening composition.

In one example, the sensate composition comprises one or more TRP receptor triggering agents (“sensates”), for example one or more TRPM8 receptor triggering agents.

In addition to the one or more TRP receptor triggering agents (“sensates”), the sensate composition may further comprise one or more TRP receptor blocking agents.

In one example, the sensate composition comprises one or more and/or two or more TRP receptor triggering agents (“sensates”), for example TRPM8 receptor triggering agents, such as menthol and menthyl lactate, and one or more and/or two or more TRP receptor blocking agents, for example a TRPA1 and TRPV1 receptor blocking agents, such as apritone and/or raspberry ketone. The TRP receptor agents (TRP receptor triggering agents and TRP receptor blocking agents) may be both in the sensate composition, may be split (one on the fibrous structure directly and the other in the sensate composition), and/or both directly on the fibrous structure.

In another example, the sensate composition comprises one or more and/or two or more TRP receptor triggering agents (“sensates”), for example TRPM8 receptor triggering agents, such as N,2,3-trimethyl-2-isopropylbutanamide (WS-23), and one or more and/or two or more TRP receptor blocking agents, such as apritone and/or raspberry ketone. The TRP receptor agents (TRP receptor triggering agents and TRP receptor blocking agents) may be both in the sensate composition, may be split (one on the fibrous structure directly and the other in the sensate composition), and/or both directly on the fibrous structure.

In another example, the sensate composition comprises one or more and/or two or more TRP receptor triggering agents (“sensates”), for example TRPM8 receptor triggering agents, such as N,2,3-trimethyl-2-isopropylbutanamide (WS-23), and one or more and/or two or more TRP receptor blocking agents, for example TRPA1, TRPV1, TRPV3 receptor blocking agents, such as laureth 9 and polyethyleneimine 2000. The TRP receptor agents (TRP receptor triggering agents and TRP receptor blocking agents) may be both in the sensate composition, may be split (one on the fibrous structure directly and the other in the sensate composition), and/or both directly on the fibrous structure.

In addition to the one or more TRPM8 receptor triggering agents and any of the additional components described herein, the sensate composition, such as a surface softening composition, may further comprise one or more additives selected from the group consisting of: surface softening agents, humectants, emollients, immobilizing agents, preservatives, and mixtures thereof.

### Surface Softening Composition

A surface softening composition, for purposes of the present invention, is a composition that improves the tactile sensation of a surface of a fibrous structure perceived by a user whom holds a fibrous structure and/or sanitary tissue product comprising the fibrous structure and rubs it across the user's skin. Such tactile perceivable softness can be characterized by, but is not limited to, friction, flexibility, and smoothness, as well as subjective descriptors, such as a feeling like lubricious, velvet, silk or flannel.

The surface softening composition may or may not be transferable. Typically, it is substantially non-transferable.

The surface softening composition may increase or decrease the surface friction of the surface of the fibrous structure, especially the user contacting surface of the fibrous structure. Typically, the surface softening composition will reduce the surface friction of the surface of the fibrous structure compared to a surface of the fibrous structure without such surface softening composition.

The surface softening composition comprises a surface softening agent. The surface softening composition during application to the fibrous structure may comprise at least about 0.1% and/or at least 0.5% and/or at least about 1% and/or at least about 3% and/or at least about 5% to 100% and/or to about 98% and/or to about 95% and/or to about 90% and/or to about 80% and/or to about 70% and/or to about 50% and/or to about 40% by weight of the surface softening agent. In one example, the surface softening composition comprises from about 5% to about 40% by weight of the surface softening agent.

The surface softening composition present on the fibrous structure and/or sanitary tissue product comprising the fibrous structure of the present invention may comprise at least about 0.01% and/or at least about 0.05% and/or at least about 0.1% of total basis weight of the surface softening agent. In one example, the fibrous structure and/or sanitary tissue product may comprise from about 0.01% to about 20% and/or from about 0.05% to about 15% and/or from about 0.1% to about 10% and/or from about 0.01% to about 5% and/or from about 0.1% to about 2% of total basis weight of the surface softening composition.

In one example, the surface softening composition may be present on and/or in the fibrous structure at a level of at least 1#/ton and/or at least 5#/ton and/or at least 10#/ton and/or at least 15#/ton.

### Surface Softening Agents

The sensate composition, for example surface softening composition may comprise one or more surface softening agents selected from the group consisting of: quaternary ammonium compounds, amines, fatty esters, sucrose esters, silicones, dispersible polyolefins, clays, polysaccharides, fatty acids, softening oils, polymer latexes, polyhydroxy compounds, and mixtures thereof.

In one example, the surface softening agent is selected from the group consisting of: quaternary ammonium compounds, silicone polymers, polysaccharides, clays, amines, fatty esters, dispersible polyolefins, polymer latex, and mixtures thereof. Non-limiting examples of suitable quaternary ammonium compounds include an alkyl quaternary ammonium compound, such as an alkyl quaternary ammonium compound selected from the group consisting of a monoalkyl quaternary ammonium compound, a dialkyl quaternary ammonium compound, a trialkyl quaternary ammonium compound, and mixtures thereof. Non-limiting examples of suitable amines include amines selected from the group consisting of: ester amines (monoester amines, diester amines, and/or triester amines), amidoesteramines, amidoamines (monoamido amines and/or diamido amines), imidazoline amines, alkyl amines (mono alkylamines, dialkyl amines quats, and/or trialkyl amines), and mixtures thereof. Non-limiting examples of suitable silicone polymers include silicone polymers selected from the group consisting of: cyclic silicones, polydimethylsiloxanes, aminosilicones, cationic silicones, silicone polyethers, silicone resins, silicone urethanes, and mixtures thereof. A non-limiting example of a suitable polysaccharide includes cationic starch. A non-limiting example of a suitable clay includes smectite clay. Non-limiting examples of suitable dispersible polyolefins include dispersible polyolefins selected from the group consisting of: polyethylene, polypropylene, and mixtures thereof. Non-limiting examples of suitable fatty esters include fatty esters selected from the group consisting of: a polyglycerol ester, a sucrose ester, a glycerol ester, and mixtures thereof.

In one example, the surface softening agent comprises a quaternary ammonium compound selected from the group consisting of ester quaternary ammonium compounds, (monoesterquats, diesterquats, and/or triesterquats), amide quaternary ammonium compounds (monoamide quats and/or diamide quats), imidazoline quaternary ammonium compounds, alkyl quaternary ammonium compounds (mono alkyl quats, dialkyl quats, trialkyl quats, and/or tetraalkyl quats), amidoester quaternary ammonium compounds, and mixtures thereof.

Non-limiting examples of suitable monoesterquats and diesterquats are selected from the group consisting of: bis-(2-hydroxypropyl)-dimethylammonium methylsulfate fatty acid ester and isomers of bis-(2-hydroxypropyl)-dimethylammonium methylsulfate fatty acid ester and/or mixtures thereof, 1,2-di(acyloxy)-3-trimethylammoniopropane chloride, N,N-bis(stearoyl-oxy-ethyl)-N,N-dimethyl ammonium chloride, N,N-bis(tallowoyl-oxy-ethyl) N,N-dimethyl ammonium chloride, bis(2-tallowoyloxyethyl)dimethyl ammonium chloride, N,N-bis(stearoyl-oxy-ethyl)-N-(2 hydroxyethyl)-N-methyl ammonium methylsulfate, N,N-bis-(stearoyl-2-hydroxypropyl)-N,N-dimethylammonium methylsulfate, N,N-bis-(tallowoyl-2-hydroxypropyl)-N,N-dimethylammonium methylsulfate, N,N-bis-(palmitoyl-2-hydroxypropyl)-N,N-dimethylammonium methylsulfate, N,N-bis-(stearoyl-2-hydroxypropyl)-N,N-dimethylammonium chloride, 1,2-di-(stearoyl-oxy)-3-trimethyl ammoniumpropane chloride, dicanoladimethylammonium chloride, di(hard)tallowdimethylammonium chloride, dicanoladimethylammonium methylsulfate, 1-methyl-1-stearoylamidoethyl-2-stearoylimidazolinium methylsulfate, 1-tallowylamidoethyl-2-tallowylimidazoline, dipalmylmethyl hydroxyethylammonium methylsulfate, and mixtures thereof.

In one example, the surface softening active has the following formula (1):

{R₄₋ₘ—N⁺—[R⁴—Y—R¹]ₘ}X⁻ (1)

wherein each R comprises either hydrogen, a short chain C₁-C₆, for example a C₁-C₃ alkyl or hydroxyalkyl group, for example methyl, ethyl, propyl, hydroxyethyl, and the like, poly(C₂₋₃ alkoxy), polyethoxy, benzyl, or mixtures thereof; each R⁴ is independently (CH₂)n, CH2-CH(CH₃)— or CH—(CH₃)—CH₂—; each Y may comprise —O—(O)C—, —C(O)—O—, —NR—C(O)—, or —C(O)—NR—; each m is 2 or 3; each n is from 1 to about 4, in one aspect 2; the sum of carbons in each R¹, plus one when Y is —O—(O)C— or —NR—C(O)—, may be C₁₂-C₂₂, for example C₁₄-C₂₂, with each R¹ being a hydrocarbyl, or substituted hydrocarbyl group; and X⁻ may comprise any softener-compatible anion. In one aspect, the softener-compatible anion may comprise chloride, bromide, methylsulfate, ethylsulfate, sulfate, and nitrate. In another example, the softener-compatible anion (X⁻) may be chloride or methyl sulfate.

An example of a suitable surface softening agent is a “propyl” ester quaternary ammonium surface softening agent, for example 1,2-di(acyloxy)-3-trimethylammoniopropane chloride.

In another example, the surface softening agent has the formula (4):

[R₄₋ₘ—N⁺—R¹ₘ]X⁻ (4)

wherein each R, R¹, m are as disclosed above, and X⁻ may comprise any softener-compatible anion. In one aspect, the softener-compatible anion may comprise chloride, bromide, methylsulfate, ethylsulfate, sulfate, and nitrate. In another example, the softener-compatible anion (X⁻) may comprise chloride or methyl sulfate.

Non-limiting examples of surface softening agents include N,N-bis(stearoyl-oxy-ethyl)-N,N-dimethyl ammonium chloride, N,N-bis(tallowoyl-oxy-ethyl)-N,N-dimethyl ammonium chloride, N,N-bis(stearoyl-oxy-ethyl)-N-(2-hydroxyethyl)-N-methyl ammonium methylsulfate.

A non-limiting example of a surface softening agent includes 1,2-di-(stearoyl-oxy)-3-trimethyl ammoniumpropane chloride.

Non-limiting examples of surface softening agents include dialkylenedimethylammonium salts such as dicanoladimethylammonium chloride, di(hard)tallowdimethylammonium chloride dicanoladimethylammonium methylsulfate, and mixtures thereof. An example of commercially available dialkylenedimethylammonium salts usable in the present invention is dioleyldimethylammonium chloride available from Witco Corporation under the trade name Adogen® 472 and dihardtallow dimethylammonium chloride available from Akzo Nobel Arquad 2HT75.

A non-limiting example of a surface softening agent includes 1-methyl-1-stearoylamidoethyl-2-stearoylimidazolinium methylsulfate available commercially from the Witco Corporation under the trade name Varisoft®.

A non-limiting example of a surface softening agent includes 1-tallowylamidoethyl-2-tallowylimidazoline.

Non-limiting examples of surface softening agents include reaction products of fatty acids with diethylenetriamine, for example in a molecular ratio of about 2:1, said reaction product mixture containing N,N″-dialkyldiethylenetriamine with the formula:

R¹—C(O)—NH—CH₂CH₂—NH—CH₂CH₂—NH—C(O)—R¹

wherein R¹ is an alkyl group of a commercially available fatty acid derived from a vegetable or animal source, such as Emersol® 223LL or Emersol® 7021, available from Henkel Corporation. In one example, the fatty acid may be obtained, in whole or in part, from a renewable source, via extraction from plant material, fermentation from plant material, and/or obtained via genetically modified organisms such as algae or yeast.

A non-limiting example of a surface softening agent includes a di-fatty amidoamine based surface softening agent having the formula:

[R¹—C(O)—NH—CH₂CH₂—N(CH₃)(CH₂CH₂OH)—CH₂CH₂—NH—C(O)—R¹]⁺CH₃SO₄⁻

wherein R¹ is an alkyl group. An example of such a surface softening agent is commercially available from the Witco Corporation e.g. under the trade name Varisoft® 222LT.

Non-limiting examples of surface softening agents include reaction products of fatty acids with N-2-hydroxyethylethylenediamine, for example in a molecular ratio of about 2:1, said reaction product mixture containing a compound of the formula:

R¹—C(O)—NH—CH₂CH₂—N(CH₂CH₂OH)—C(O)—R¹

wherein R¹—C(O) is an alkyl group of a commercially available fatty acid derived from a vegetable or animal source, such as Emersol® 223LL or Emersol® 7021, available from Henkel Corporation.

A non-limiting example of a surface softening agent includes a dialkyl imidazoline diester compound, where the compound is the reaction product of N-(2-hydroxyethyl)-1,2-ethylenediamine or N-(2-hydroxyisopropyl)-1,2-ethylenediamine with glycolic acid, esterified with fatty acid, where the fatty acid is (hydrogenated) tallow fatty acid, palm fatty acid, hydrogenated palm fatty acid, oleic acid, rapeseed fatty acid, hydrogenated rapeseed fatty acid or a mixture of the above.

In one example, the surface softening agent is selected from the group consisting of: ditallowoyloxyethyl dimethyl ammonium chloride, dihydrogenated-tallowoyloxyethyl dimethyl ammonium chloride, ditallow dimethyl ammonium chloride, dihydrogenatedtallow dimethyl ammonium chloride, ditallowoyloxyethyl methylhydroxyethylammonium methyl sulfate, dihydrogenated-tallowoyloxyethyl methyl hydroxyethylammonium chloride, and mixtures thereof.

As used herein, the term “polyhydroxy compound(s)” is defined as a chemical agent that imparts lubricity or emolliency to tissue paper products and also possesses permanence with regard to maintaining the fidelity of its deposits without substantial migration when exposed to the environmental conditions to which products of this type are ordinarily exposed during their typical life cycle. The present invention contains as an essential component from about 2.0% to about 30.0%, preferably from 5% to about 20.0%, more preferably from about 8.0% to about 15.0%, of a water soluble polyhydroxy compound, based on the dry fiber weight of the tissue paper.

Non-limiting examples of water soluble polyhydroxy compounds suitable for use in the present invention include glycerol, polyglycerols having a weight average molecular weight of from about 150 to about 800 and polyoxyethylene glycol and polyoxypropylene glycol having a weight-average molecular weight of from about 200 to about 4000, preferably from about 200 to about 1000, most preferably from about 200 to about 600. Polyoxyethylene glycol having a weight average molecular weight of from about 200 to about 600 are especially preferred. Mixtures of the above-described polyhydroxy compounds may also be used. For example, mixtures of glycerol and polyglycerols, mixtures of glycerol and polyoxyethylene glycols, ‘mixtures of polyglycerols and polyoxyethylene glycols/oxides, etc. are useful in the present invention. A particularly preferred polyhydroxy compound is polyoxyethylene glycol having a weight average molecular weight of about 200. This material is available commercially from The Dow Chemical Company under the trade name “Carbowax™ 200.”

Specific examples of ester-functional quaternary ammonium compounds having the structures detailed above and suitable for use in the present invention may include the diester dialkyl dimethyl ammonium salts such as diester ditallow dimethyl ammonium chloride, monoester ditallow dimethyl ammonium chloride, diester ditallow dimethyl ammonium methyl sulfate, diester di(hydrogenated)tallow dimethyl ammonium methyl sulfate, diester di(hydrogenated)tallow dimethyl ammonium chloride, and mixtures thereof, for example diester ditallow dimethyl ammonium chloride and/or diester di(hydrogenated)tallow dimethyl ammonium chloride, which are commercially available from Witco Chemical Company Inc. of Dublin, Ohio under the tradename “ADOGEN SDMC”.

The surface softening agent may exhibit an Iodine Value of between 0-140 and/or from about 5 to about 100 and/or from about 10 to about 80 and/or from about 15 to about 70 and/or from about 18 to about 60 and/or from about 18 to about 25. In one example, when the surface softening agent is a partially hydrogenated fatty acid quaternary ammonium compound it exhibits a Iodine Value of from about 25 to about 60.

In one example, the sensate composition, for example surface softening composition of the present invention comprises two or more different surface softening agents, for example two or more different quaternary ammonium compounds and/or at least one quaternary ammonium compound and at least one silicone polymer. When the sensate composition, for example surface softening composition comprises one or more surface softening agents, for example a quaternary ammonium compound and/or a silicone polymer, the surface softening agent may be present in the sensate composition at a level of from about 0.001% to about 10% and/or from about 0.1% to about 8% and/or from about 0.5% to about 5% by weight.

Non-limiting examples of suitable surface softening agents that can be present in the surface softening composition of the present invention can be selected from the group consisting of: polymers such as polyethylene and derivatives thereof, hydrocarbons, waxes, oils, silicones, organosilicones (oil compatible), quaternary ammonium compounds, fluorocarbons, substituted C₁₀-C₂₂ alkanes, substituted C₁₀-C₂₂ alkenes, in particular derivatives of fatty alcohols and fatty acids (such as fatty acid amides, fatty acid condensates and fatty alcohol condensates), polyols, derivatives of polyols (such as esters and ethers), sugar derivatives (such as ethers and esters), polyglycols (such as polyethyleneglycol), and mixtures thereof.

In one example, the surface softening composition of the present invention is a microemulsion and/or a macroemulsion of a surface softening agent (for example an aminofunctional polydimethylsiloxane, specifically an aminoethylaminopropyl polydimethylsiloxane) in water. In such an example, the concentration of the surface softening agent within the surface softening composition may be from about 3% to about 60% and/or from about 4% to about 50% and/or from about 5% to about 40%. Non-limiting examples of such microemulsions are commercially available from Wacker Chemie (MR1003, MR103, MR102). A non-limiting example of such a macroemulsion is commercially available from Momentive, Columbus, Ohio (CM849).

Non-limiting examples of suitable waxes may be selected from the group consisting of: paraffin, polyethylene waxes, beeswax, and mixtures thereof.

Non-limiting examples of suitable oils may be selected from the group consisting of: mineral oil, silicone oil, silicone gels, petrolatum, and mixtures thereof.

Non-limiting examples of suitable silicones may be selected from the group consisting of: polydimethylsiloxanes, aminosilicones, cationic silicones, quaternary silicones, silicone betaines, and mixtures thereof.

In one example, the surface softening agent comprises a partially hydrogenated tallow diester chloride quaternary ammonium compound, for example bis(2-tallowoyloxyethyl)dimethyl ammonium chloride, premixed with polyethylene glycol, for example PEG 400. For example, the premix is about 70-75% quaternary ammonium compound (such as Adogen SDMC-type from Witco Corporation and 25-30% PEG 400, available from J. T. Baker Company of Phillipsburg, N.J.).

The surface softening composition may comprise additional ingredients such as a vehicle as described herein below which may not be present on the fibrous structure and/or sanitary tissue product comprising such fibrous structure. In one example, the surface softening composition may comprise a surface softening agent and a vehicle such as water to facilitate the application of the surface softening agent onto the surface of the fibrous structure.

It is understood that combinations of any of the surface softening agents disclosed herein are suitable for use in the surface softening compositions of the present invention.

### NON-LIMITING EXAMPLES

### Example 1

A first stock chest of 100% eucalyptus fiber is prepared with a conventional pulper to have a consistency of about 3.0% by weight. The thick stock of the first hardwood chest is directed through a thick stock line where a wet-strength additive, HERCOBOND 1194 (commercially available from Ashland Inc.), is added in-line to the thick stock at about 0.5 lbs. per ton of dry fiber as it moves to the first fan pump.

Additionally, a second stock chest of 100% eucalyptus fiber is prepared with a conventional pulper to have a consistency of about 3.0% by weight. The thick stock of the second hardwood chest is directed through a thick stock line where a wet-strength additive, HERCOBOND 1194, is added in-line to the thick stock at about 0.5 lbs. per ton of dry fiber as it moves to the second fan pump.

A third stock chest is prepared with 100% NSK fiber with a final consistency of about 3.0%. The blended thick stock is directed to a disk refiner where it is refined to a Canadian Standard Freeness of about 580 to 625. The NSK thick stock of the third stock chest is then directed through a thick stock line where a wet-strength additive, HERCOBOND 1194, is added to the thick stock at about 1.5 lbs. per ton of dry fiber. The refined, 100% NSK thick stock is then directed to a third fan pump.

The eucalyptus fiber slurry diluted by the first fan pump is directed through the bottom headbox chamber (Yankee-side layer). The NSK fiber slurry diluted by the third fan pump is directed through the center headbox chamber. The eucalyptus fiber slurry diluted by the second fan pump directed to the top headbox chamber (Fabric-side) and delivered in superposed relation to the fixed-roof former's forming wire to form thereon a three-layer embryonic web, of which about 34.5% of the top side is made up of pure eucalyptus fibers, center is made up of about 34.5% of a NSK fiber and the bottom side (Yankee-side) is made up of about 34.5% of pure eucalyptus fiber. Dewatering occurs through the outer wire and the inner wire and is assisted by wire vacuum boxes. Forming wire is an 84M design traveling at a speed of 800 fpm (feet per minute).

The embryonic wet web is transferred from the carrier (inner) wire, at a fiber consistency of about 24% at the point of transfer, to a patterned drying fabric. The speed of the patterned drying fabric is about 800 fpm (feet per minute). The drying fabric is designed to yield a pattern of substantially machine direction oriented linear channels having a continuous or semi-continuous network of high density (knuckle) areas. This drying fabric is formed by casting an impervious resin surface onto a fiber mesh supporting fabric. The supporting fabric is a 127×52 filament, dual layer mesh. The thickness of the resin cast is about 12 mils above the supporting fabric.

While remaining in contact with the patterned drying fabric, the web is pre-dried by air blow-through pre-dryers to a fiber consistency of about 60% by weight.

After the pre-dryers, the semi-dry web is transferred to the Yankee dryer through a nip formed by the pressure roll surface and the Yankee surface where the Yankee surface has been pre-treated with a sprayed a creping adhesive coating. The coating is a blend consisting of Georgia Pacific's UNICREPE 457T20 and Vinylon Works' VINYLON 8844 at a ratio of about 92 to 8, respectively. The fiber consistency is increased to about 97% before the web is dry creped from the Yankee with a doctor blade.

The web is removed from the Yankee surface by a creping blade having a bevel angle of about 25 degrees and is positioned with respect to the Yankee dryer to provide an impact angle of about 81 degrees. The Yankee dryer is operated at a temperature of about 350° F. (177° C.) and a speed of about 800 fpm. The fibrous structure is wound in a roll using a surface driven reel drum having a surface speed of about 700 fpm (feet per minute) to make a parent roll.

During a converting process, one or more TRP receptor triggering agents (“sensates”), for example one or more TRPM8 receptor triggering agents, described herein, alone or in combination with one or more TRP receptor blocking agents described herein in the form of a sensate composition, such as a surface softening composition, is applied, for example with a slot extrusion die, to the outside surface of the fibrous structure as the parent roll is unwound and converted into a finished fibrous structure and/or sanitary tissue product. If a multi-ply sanitary tissue product is desired, then two or more of the fibrous structure plies or at least one with a different fibrous structure ply can be bonded together to form a multi-ply sanitary tissue product, for example a multi-ply toilet tissue.

### Example 2

A first stock chest of 100% eucalyptus fiber is prepared with a conventional pulper to have a consistency of about 3.0% by weight. The thick stock of the first hardwood chest is directed through a thick stock line where a permanent wet-strength additive, such as KYMENE™ (commercially available from Solenis), is added in-line to the thick stock at about 0.5 lbs. per ton of dry fiber as it moves to the first fan pump.

Additionally, a second stock chest of 100% eucalyptus fiber is prepared with a conventional pulper to have a consistency of about 3.0% by weight. The thick stock of the second hardwood chest is directed through a thick stock line where a permanent wet-strength additive, such as KYMENE™ (commercially available from Solenis), is added in-line to the thick stock at about 0.5 lbs. per ton of dry fiber as it moves to the second fan pump.

A third stock chest is prepared with 100% NSK fiber with a final consistency of about 3.0%. The blended thick stock is directed to a disk refiner where it is refined to a Canadian Standard Freeness of about 580 to 625. The NSK thick stock of the third stock chest is then directed through a thick stock line where a permanent wet-strength additive, such as KYMENE™ (commercially available from Solenis), is added to the thick stock at about 1.5 lbs. per ton of dry fiber. The refined, 100% NSK thick stock is then directed to a third fan pump.

The eucalyptus fiber slurry diluted by the first fan pump is directed through the bottom headbox chamber (Yankee-side layer). The NSK fiber slurry diluted by the third fan pump is directed through the center headbox chamber. The eucalyptus fiber slurry diluted by the second fan pump directed to the top headbox chamber (Fabric-side) and delivered in superposed relation to the fixed-roof former's forming wire to form thereon a three-layer embryonic web, of which about 34.5% of the top side is made up of pure eucalyptus fibers, center is made up of about 34.5% of a NSK fiber and the bottom side (Yankee-side) is made up of about 34.5% of pure eucalyptus fiber. Dewatering occurs through the outer wire and the inner wire and is assisted by wire vacuum boxes. Forming wire is an 84M design traveling at a speed of 800 fpm (feet per minute).

The embryonic wet web is transferred from the carrier (inner) wire, at a fiber consistency of about 24% at the point of transfer, to a patterned drying fabric. The speed of the patterned drying fabric is about 800 fpm (feet per minute). The drying fabric is designed to yield a pattern of substantially machine direction oriented linear channels having a continuous or semi-continuous network of high density (knuckle) areas. This drying fabric is formed by casting an impervious resin surface onto a fiber mesh supporting fabric. The supporting fabric is a 127×52 filament, dual layer mesh. The thickness of the resin cast is about 12 mils above the supporting fabric.

While remaining in contact with the patterned drying fabric, the web is pre-dried by air blow-through pre-dryers to a fiber consistency of about 60% by weight.

After the pre-dryers, the semi-dry web is transferred to the Yankee dryer through a nip formed by the pressure roll surface and the Yankee surface where the Yankee surface has been pre-treated with a sprayed a creping adhesive coating. The coating is a blend consisting of Georgia Pacific's UNICREPE 457T20 and Vinylon Works' VINYLON 8844 at a ratio of about 92 to 8, respectively. The fiber consistency is increased to about 97% before the web is dry creped from the Yankee with a doctor blade.

The web is removed from the Yankee surface by a creping blade having a bevel angle of about 25 degrees and is positioned with respect to the Yankee dryer to provide an impact angle of about 81 degrees. The Yankee dryer is operated at a temperature of about 350° F. (177° C.) and a speed of about 800 fpm. The fibrous structure is wound in a roll using a surface driven reel drum having a surface speed of about 700 fpm (feet per minute) to make a parent roll.

During a converting process, one or more TRP receptor triggering agents (“sensates”), for example one or more TRPM8 receptor triggering agents, described herein, alone or in combination with one or more TRP receptor blocking agents described herein in the form of a sensate composition, such as a surface softening composition, is applied, for example with a slot extrusion die, to the outside surface of the fibrous structure as the parent roll is unwound and converted into a finished fibrous structure and/or sanitary tissue product. If a multi-ply sanitary tissue product is desired, then two or more of the fibrous structure plies or at least one with a different fibrous structure ply can be bonded together to form a multi-ply sanitary tissue product, for example a multi-ply facial tissue.

### Example 3

The individual plies of this Example 3 are made according to the process detailed in Example 1 above. Two plies were combined with the wire side facing out. During the converting process, a surface softening agent and optionally, a lotion are applied sequentially, if both are present, with one or more slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 14.1% by weight and the lotion, when present, is applied to the web at a rate of 5.0% by weight. The plies are then bonded together with plybond glue and then wound into a finished 2-ply toilet tissue product. Each user unit tested in accordance with the test methods described above.

### Example 4

The individual plies of this Example 4 are made according to the process detailed in Example 2 above. Two plies were combined with the wire side facing out. During the converting process, a surface softening agent and a lotion are applied sequentially with slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 14.1% by weight and the lotion is applied to the web at a rate of 5.0% by weight. The plies are then bonded together with mechanical ply-bonding wheels, slit, and then folded into finished 2-ply facial tissue product. Each user unit tested in accordance with the test methods described above.

### Example 5

The individual plies of this Example 5 are made according to the process detailed in Example 2 above. Two plies were combined with the wire side facing out. During the converting process, a surface softening agent and a lotion are applied sequentially with slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 10.0% by weight and the lotion is applied to the web at a rate of 5.0% by weight. The plies are then bonded together with mechanical ply-bonding wheels, slit, and then folded into finished 2-ply facial tissue product. Each user unit tested in accordance with the test methods described above.

### Example 6

The individual plies of this Example 6 are made according to the process detailed in Example 2 above. Two plies were combined with the wire side facing out. During the converting process, a surface softening agent and a lotion are applied sequentially with slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 10.0% by weight and the lotion is applied to the web at a rate of 10.4% by weight. The plies are then bonded together with mechanical ply-bonding wheels, slit, and then folded into finished 2-ply facial tissue product. Each user unit tested in accordance with the test methods described above.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A fibrous structure comprising a sensate composition comprising one or more TRPM8 receptor triggering agents and at least one TRP receptor blocking agent.

2. The fibrous structure according to claim 1 wherein the sensate composition is present on a user contacting surface of the fibrous structure.

3. The fibrous structure according to claim 1 wherein at least one of the TRPM8 receptor triggering agents is selected from the group consisting of: carboxamides, ketals, esters, ethers, alcohols, and mixtures thereof.

4. The fibrous structure according to claim 3 wherein at least one of the TRPM8 receptor triggering agents is N,2,3-trimethyl-2-isopropylbutanamide.

5. The fibrous structure according to claim 1 wherein the sensate composition is void of TRPM8 receptor triggering agents selected from the group consisting of: esters of menthol and menthol derivatives, menthylcarboxylic acid ester derivatives, carboxamides, cyclohexane carboxy ester derivatives, cyclohexylmethyl carboxylate derivatives, cyclohexyl anthranilate derivatives, omega-amino cyclohexyl carboxylate derivatives, omega-amino cyclohexane carboxy ester derivatives, omega-amino cyclohexylmethyl carboxylate derivatives, camphor, and mixtures thereof.

6. The fibrous structure according to claim 1 wherein the sensate composition is void of TRPM8 receptor triggering agents selected from the group consisting of: menthol, camphor, and mixtures thereof.

7. The fibrous structure according to claim 1 wherein the fibrous structure elicits a response from a TRPM8 receptor within 30 seconds of contact with a surface.

8. The fibrous structure according to claim 1 wherein the fibrous structure elicits a response from a TRPM8 receptor that exhibits a duration of less than 1 minute.

9. The fibrous structure according to claim 1 wherein at least one of the TRPM8 receptor triggering agents is transferable to a surface.

10. The fibrous structure according to claim 9 wherein the surface is a user's skin.

11. The fibrous structure according to claim 10 wherein the user's skin comprises a user's anal skin.

12. The fibrous structure according to claim 1 wherein the one or more TRPM8 receptor triggering agents are present on the fibrous structure at a level of greater than 0% to less than about 3% by weight of the fibrous structure.

13. The fibrous structure according to claim 1 wherein no TRPM8 receptor triggering agents are present on the fibrous structure at a level of greater than 3% by weight of the fibrous structure.

14. The fibrous structure according to claim 1 wherein at least one of the TRPM8 receptor triggering agents is present in the sensate composition at a level of at least 0.25% by weight of the sensate composition.

15. The fibrous structure according to claim 1 wherein at least one of the TRP receptor blocking agents blocks one or more of the TRP receptors selected from the group consisting of TRPA1 receptor, TRPV receptor, TRPV1 receptor, TRPA1V1 receptor, TRPV2 receptor, TRPV3 receptor, TRPV4 receptor, and mixtures thereof.

16. The fibrous structure according to claim 1 wherein at least one of the TRP receptor blocking agents blocks two or more TRP receptors.

17. The fibrous structure according to claim 1 wherein at least one of the TRP receptor blocking agents is a selective TRP receptor blocking agent.

18. The fibrous structure according to claim 1 wherein at least one of the TRP receptor blocking agents is a reversible TRP receptor blocking agent.

19. The fibrous structure according to claim 1 wherein the sensate composition further comprises an additive selected from the group consisting of: surface softening agents, humectants, emollients, immobilizing agents, preservatives, and mixtures thereof.

20. A single- or multi-ply sanitary tissue product comprising a fibrous structure according to claim 1.
